(19) United States
(12) Patent Application Publication
(10) Pub. No.: US 2017/0105863 A1
(43) Pub. Date: Apr. 20, 2017

(54) Full Contact Orthopedic Cast and Method

(71) Applicant: WoundKair Concepts, Inc., Benbrook, TX (US)

(72) Inventors: Danny E. Anderson, Aledo, TX (US); Kimberly G. Anderson, Aledo, TX (US); Glenn T. Carlson, Keller, TX (US)

(21) Appl. No.: 15/291,566

(22) Filed: Oct. 12, 2016

Related U.S. Application Data
(60) Provisional application No. 62/242,026, filed on Oct. 15, 2015.

(51) Int. Cl. A61F 5/01; A61F 13/06; A61F 13/04

(52) U.S. Cl. CPC A61F 5/0111; A61F 13/048; A61F 13/045; A61F 13/064

(57) ABSTRACT

A fully conformable, full contact orthopedic cast is shown for an affected area of a patient such as a limb or appendage requiring immobilization. A method for installing the cast on a patient is also shown. The cast includes an initially flexible bladder which is filled with a curable compound. The bladder may be conformably installed on the limb to be immobilized. Once the bladder is conformed to the limb, the curable compound is cured, as by the use of LED light, or UV-irradiation. The cast is easy to remove simply by unzipping a cross-linked closure strip, eliminating the need to use a saw to remove the cast.

### CROSS-REFERENCE TO RELATED APPLICATIONS

The present application claims priority from an earlier filed provisional application Ser. No. 62/242,026, filed Oct. 15, 2015, entitled “Orthopedic Cast”, by the same inventors.

### BACKGROUND OF THE INVENTION

1. Field of the Invention

The present invention generally relates to the field of orthopedic bandages, splints, and casts and more particularly to fully conformable “full contact” orthopedic casts that do not require the use of a saw to remove them.

2. Background of the Invention and Description of the Prior Art

An orthopedic cast, body cast, plaster cast, or surgical cast, is a shell, frequently made from plaster or fiberglass, encasing a limb (or, in some cases, large portions of the body) to stabilize and hold anatomical structures, most often a broken bone (or bones), in place until healing is achieved. Orthopedic casts made of Plaster of Paris have a long history of use because of their ability to immobilize a broken bone or other portion or appendage of a body while healing takes place. Plaster of Paris casts are formed using a fabric tape such as cotton muslin that is impregnated with a solution of calcium sulfate. The cast hardens as the material dries, forming a rigid shell around the site of the injury. A more recent substitute for the Plaster of Paris is the use of fiberglass tape impregnated with a synthetic resin. A fiberglass cast is also an effective mobilizer, is lighter than a plaster cast, and takes less time to apply and harden.

A specialized type of orthopedic cast, referred to herein as a “full contact orthopedic cast” or “total contact cast” (TCC) is is a specially designed cast designed to take weight off the foot (off-loading) and is particularly useful in treating patients with diabetic foot ulcers (DFUs), Reducing pressure on the wound by taking weight of the foot has proven to be very effective in DFU treatment. DFUs are a major factor leading to lower leg amputations among the diabetic population in the United States. TCC has been used for off-loading DFUs in the US since the mid-1960s and is regarded by many practitioners as the “reference standard” for off-loading the bottom surface (sole) of the foot.

TCC involves encasing the patient's complete foot, including toes, and the lower leg in a specialist cast that redistributes weight and pressure in the lower leg and foot during everyday movements. Materials used in applying the cast have changed over the years, from the use of unmodified Plaster of Paris, to formulations containing fiberglass and other materials. Fiberglass has certain advantages over Plaster of Paris, which can take too long to fully dry, among other disadvantages. The curing time of a fiberglass cast is far shorter than plaster of Paris, sometimes allowing the patient to walk with an outer boot within an hour of application.

While effective for their particular intended purposes, both of these traditional types of cast construction have several substantial disadvantages. They require significant amounts of time to apply, they are sometimes heavy, they are often not fully conformable, and they are difficult to remove, requiring the use of a saw to cut through the hardened shell of the cast. Forming a traditional cast requires applying layer upon layer of fabric tape soaked in the calcium sulfate solution or resin. This is a time-consuming step that further requires a substantial amount of time to set and harden before the patient can use the limb encased in the cast. The traditional cast typically leaves voids within its hardened shell, which prevents the cast from providing uniform, full-contact support. The lack of uniform support can lead to inflamation, abrasions, dermatitis, ulceration, and even infection, depending on the type injury requiring the application of a cast to immobilize the patient's limb, foot, etc. Moreover, the use of a saw to cut through the cast requires substantial skill in its use to avoid injury to the patient. Further, the use of a saw is often distressing to the patient—especially children—and the care-giver because of its noise and potential for injury.

What is needed is a cast design that is easy to apply, provides the required immobilization, and is easy and safe to remove, as well as economical for the patient and the caregivers.

### SUMMARY OF THE INVENTION

In one embodiment of the invention a fully conformable, full contact orthopedic cast for a limb or appendage requiring immobilization is disclosed, In one illustrative embodiment of the practice of the invention, a stockinette is first applied to the limb or appendage; a fabric cast padding material is applied over the stockinette; a system of felt strips is applied to the outside of the stockinette to provide structural form to the finished cast. Next, a flexible bladder is provided which is formed of a synthetic sheet material which is bonded around the perimeter of its edges. A locking closure strip is disposed along at least selected edges of the bladder and preferably joins one end of the bladder to its opposite end. A curable liquid, dry or clay compound is applied to fill the bladder to a predetermined level. The bladder is wrapped around the affected appendage over the felt strips and over the stockinette. Where the curable compound is a liquid, the bladder is typically provided with a sealable opening for filling. The curable compound is then cured using an appropriate source of energy or curing agent. For example, where the curable compound is a curable liquid resin, the energy source may be irradiating energy, one example being UV-energy. The curable compound may also be one of the newer so-called LED light curable compounds.

While the above summary briefly describes a preferred form of the invention, it will be understood that the stockinette, felt strips and padding may or may not be used . Also, the stockinette, felt strips and padding, where used, may be incorporated into or attached to the final design of the bladder.

In another embodiment, a method of forming a fully conformable orthopedic cast for a limb or appendage requiring immobilization is disclosed where any necessary wound dressing is first applied to the affected appendage. A soft fabric sleeve, such as a stockinette, may be then applied over the region to be enclosed in the cast. The sleeve may then be covered with cast padding material, including felt strips as needed for structural support of the cast. A cast structure, in the form of a pre- or post- filled bladder, as previously described is then provided. The bladder is filled to a desired level with a curable compound which may be a liquid, dry or clay compound, or may include other curable compounds capable of being installed within the bladder. In one exemplary embodiment, the curable compound is a curable resin compound which is installed within the bladder. After the bladder is shaped appropriately to the contours of the affected appendage, the curable compound within the bladder is cured, allowing the material within the bladder to harden. In some cases, the principles of the invention might even be used in applying a body cast.

In either embodiment of the invention described, a walking sole may be attached to the finished cast when installed on a person's foot.

### BRIEF DESCRIPTION OF THE DRAWINGS

FIG. 1 illustrates a lower leg of a patient with a stockinette applied, after any wound is cleaned and dressed;

FIG. 2 illustrates the addition of a foam toe form applied over the stockinette;

FIG. 3 illustrates application of strips of adhesive-backed felt padding to form a frame, applied to both sides of the appendage and, if a lower leg, along the tibia;

FIG. 4 illustrates, in simplified fashion, the steps of wrapping the bladder around the limb and joining it with the intermediate panel in place along the front side of the limb, and securing the cross-linked closure strips between the adjoining edges of the bladder and intermediate panel;

FIG. 5 illustrates a cross-section of the isolated bladder where the space between the outer and inner layers or sides of the bladder is filled with a curable material that is cured after the bladder is installed and shaped by hand to conform it to the patient's limb or appendage;

FIG. 6 is a simplified front view of the isolated bladder showing the location of the cross-linked locking closure that secures the intermediate panel to the forward edges of the bladder;

FIG. 7 is a simplified front end view of the isolated bladder and detail of the cross-linked locking closure on either side of the intermediate panel;

FIGS. 7A and 7B are close-up, isolated views of the regions 7A and 7B in FIG. 7;

FIG. 8 illustrates a bottom view of the walking surface or sole component of the cast;

FIG. 9 illustrates an end view of the walking surface or sole component of FIG. 8;

FIG. 10 illustrates a top view of the sole component.

FIG. 11 is an enlarged view of FIG. 3.

### DETAILED DESCRIPTION OF THE INVENTION

An advance in the state of the art is disclosed that provides a fully conformable, full contact orthopedic cast that is both easy to apply and easy to remove without the use of a saw. A conformable, initially flexible synthetic bladder, equipped with a cross-linked closure mechanism, replaces the heavy, rigid, poorly conforming cast shell formed by wrapping plaster-soaked fabric or resin-impregnated fiberglass fabric around a limb or other appendage to be encased within a cast following injury or surgery. The bladder is filled to a selected level with a curable compound which can be cured once the bladder is shaped to the appendage of the patient. A plantar support or walking surface, tread or sole can be easily installed with the bladder or added after the curable material of the cast has hardened.

The fully conformable cast, sometimes referred to as the total contact cast (ICC), means that the body of the cast is in full and even conformance with the surface of the encased body part, providing maximum support and uniform distribution of forces and pressures that act on the encased limb, while minimizing any voids inside the cast structure that impair the uniform distribution of pressure to the encased limb. Such voids can lead to abrasions and other localized injuries that complicate the healing and recovery processes enabled by having the cast in place on a patient's limb. For example, a cast installed on a person's foot, ankle, and lower leg—a “short leg cast,” must adapt to many contours due to the shape of the foot, its arch and ankle, any deformities that may be present in an individual, and the like, without exerting undue pressure upon any portion of the encased part, particularly any surface wound inside the cast.

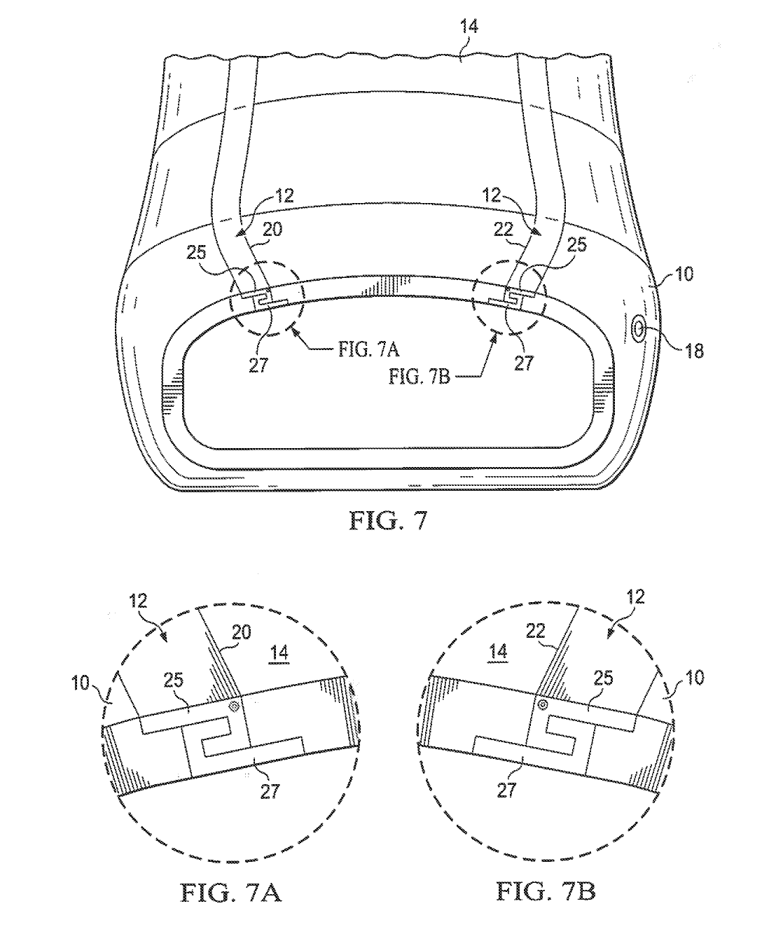

The initially flexible bladder (10 in FIG. 7) is supplied as an open wrap with an cross-linked closure 12 extending—usually lengthwise—from one end of the appendage or limb to be encased to its opposite end. This configuration enables the bladder to be wrapped around the limb to be encased in the cast until the edges of the closure can be brought together and secured with the cross-linked closure. The bladder itself is formed of first and second identical panels that are joined at their respective edges around the perimeter, in the manner of a hot water bottle sized to fit around the limb or other body part to be encased in the cast. The panels may preferably be elastomeric such as natural or synthetic rubber. In the simplest form, two of the opposite edges may be brought together and secured with the cross-linked closure (12 in FIG. 7). In some applications, an intermediate panel (14 in FIG. 7) may be used between the edges of the bladder to be joined. Where the intermediate bladder is used, a pair of cross-lined closures are used to secure the bladder in place. The intermediate panel 14 may be used to facilitate cast removal or to accommodate a larger appendage.

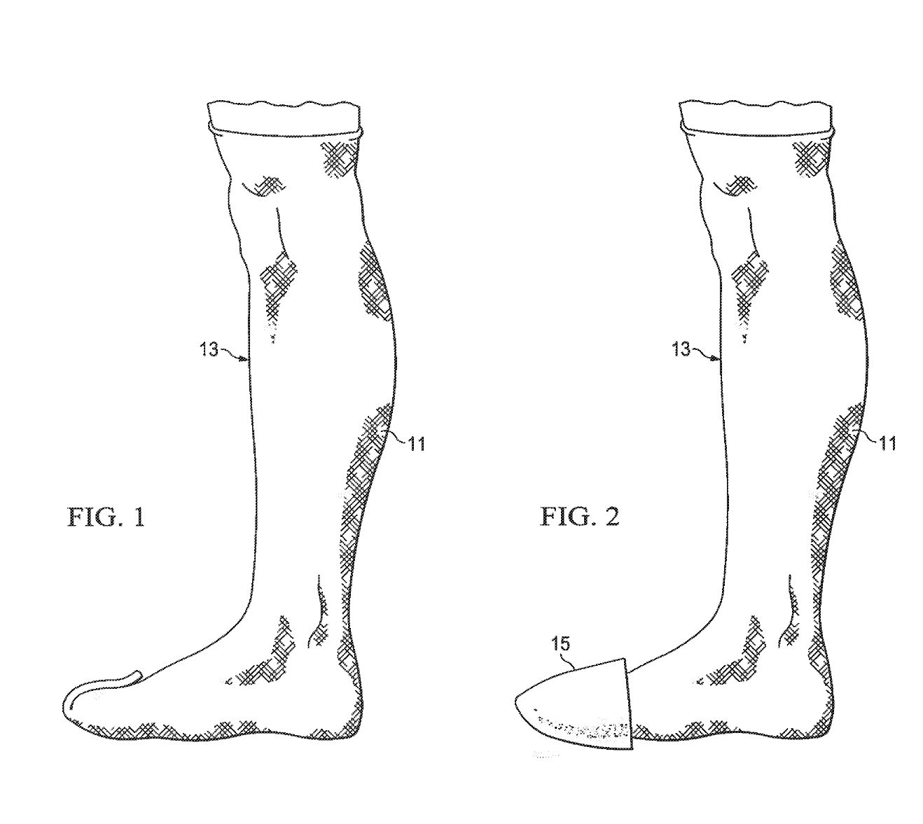

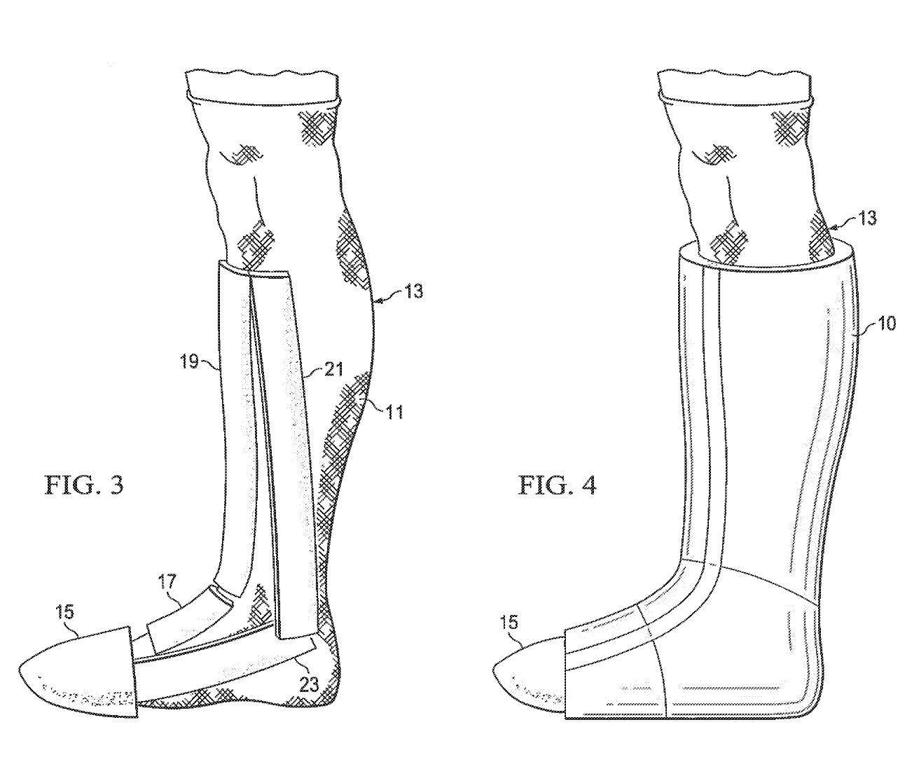

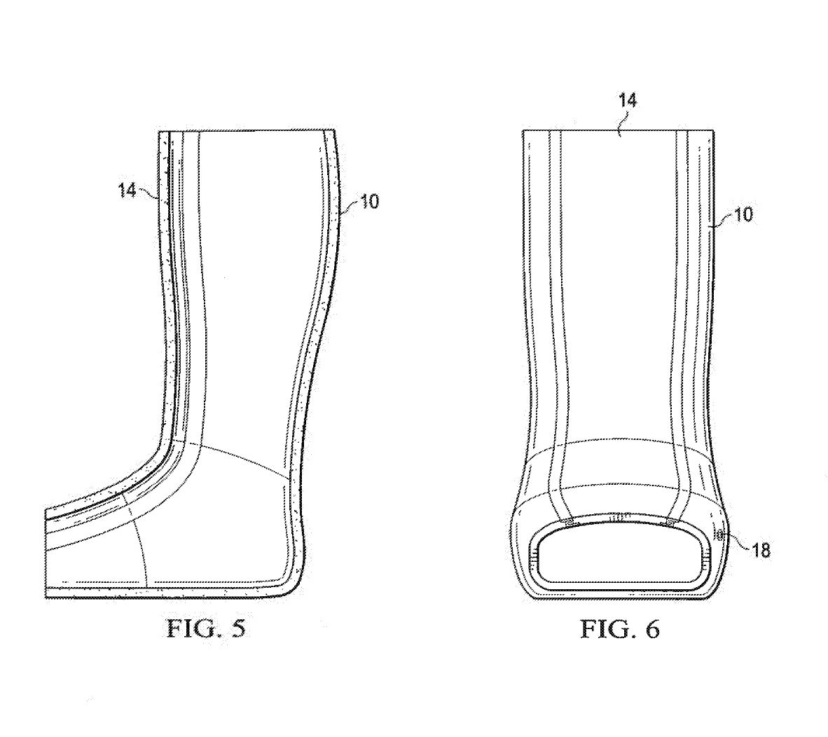

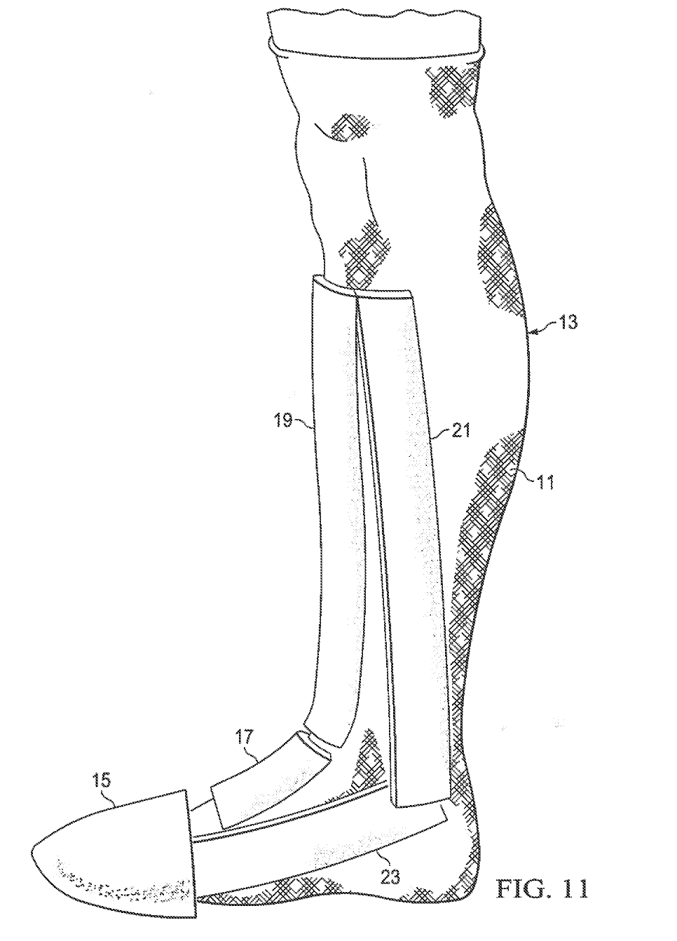

As briefly discussed, and illustrated in simplified fashion in FIGS. 1-3, certain preliminary steps may accompany the application of the flexible bladder to the affected appendage of the patient. FIG. 1 illustrates the application of a stockinette 11 to the lower leg region 13 of a patient, after any wound is cleaned and dressed. FIG. 2 illustrates the addition of a foam toe form 15 applied over the stockinette 11. FIG. 3 then illustrates the application of strips of adhesive-backed felt padding to form a frame, applied to both sides of the appendage and, if a lower leg, along the tibia. These strips, can conveniently be provided as a non-woven felt material on the order of 0.250 inch thick. The felt material may be made of synthetic fibers such as rayon or polyester, or natural fibers such as wool or cotton. Blended fibers may also be used. These steps may be easier to appreciate from the enlarged view shown in FIG. 11. The toe foam is shown as 15. A talus pad 17 is also shown, together with the tibia crest felt strip 19 and lateral and medial felt strip 21. A side foot felt strip 23 is also used in this application.

These pre-casting steps are discussed, because they are typical of the steps normally employed in installed the traditional orthopedic cast, However, as has been mentioned, these steps merely describe one form of the practice of the present invention and it will be understood that the stockinette, felt strips and padding may or may not be used Also, the stockinette, felt strips and padding, where used, may be incorporated into or attached to the final design of the bladder.

FIG. 7 shows the details of one possible cross-linked closure which may be used in the practice of the invention. Here, first and second elongated strips 25, 27, are provided having a hook-shaped cross section (at right angles to the longitudinal axis of the strips) that function somewhat like a zipper fastener, as shown in FIG. 7. The initially flexible bladder is installed, as shown in FIG. 4, with the curable material either pre- or post-installed within the interior of the bladder 10. Either before or after the bladder 10 is installed, the curable compound is deposited inside the bladder and the bladder is shaped by hand around the encased limb or appendage until it conforms to the encased limb or appendage. As will be described, forming the bladder may require working or massaging the shape to conform it to the encased limb in a uniform, comfortable way that supports the limb in the proper position. When the bladder 10 fully conforms to the encased part, the curing or hardening of the curable compound inside the interior of the bladder is initiated.

When the cast is to be removed, a special tool may be inserted between the two strips of the cross-linked closure (25, 27 in FIG. 7) to separate them and enable removal of the hardened cast. The special tool does not have a saw blade or other sharp, moving part that could cause injury if improperly used. The removal tool acts to pry apart the two sides of the cross-linked closure strips. The removal tool acts in the nature of an “unlock key.” One of the attributes of the unlock key is “forced compliance” of the patient. In other words, the “crossed-linked enclosure”, along with the unlock key, better assure that the patient may not remove, or self-adjust or otherwise alter, the full contact fit of the appliance. The “disengagement features” of the cast of the invention allow for the removal of the cast without the need or requirement of a saw or other sharp secondary device.

Installing the bladder cast itself may proceed as follows: wrapping the bladder cast around the appendage to be enclosed in the cast; (as an alternate step) providing a secondary, elongated, intermediate bladder panel having first and second substantially parallel edges forming a tongue along and between first and second edges of the bladder and having first and second strips of the locking closure disposed along the first and second edges thereof; and shaping the cast to conform the bladder cast to the enclosed appendage. Following the shaping step, hardening the conformed bladder filled with the curable compound may proceed.

As has been briefly described, the bladder 10 itself is preferably formed of a flexible, non-porous, synthetic or natural rubber sheet material having first and second sides that are sealed along the edges thereof. The synthetic sheet material is preferably capable of being hot-welded along the edges thereof, for example using an ultrasonic or radio frequency heating process. Other manufacturing techniques may also be utilized in some circumstances, such as blow-molding or other seamless types of manufacturing processes. The two-part locking closure (called the “cross-linked closure” herein) is disposed along edges of the bladder to be joined from one end of the bladder to its opposite end. The bladder should have a sealable port (such as port 18 in FIG. 7) for filling the bladder with the curable compound where a liquid curable compound is used. As discussed, a secondary, elongated, intermediate bladder panel(14 in FIG. 7) having first and second substantially parallel edges (20, 22) may be disposed along and between first and second edges of the bladder and having first and second strips of the locking closure disposed along the first and second edges thereof. The intermediate panel 14 essentially forms a tongue that facilitates securing the edges of the bladder together, and may further facilitate removal of the cast.

The curable compound may take the form of an initially liquid or viscous curable resin compound that is installed in liquid form into a sealable opening or port (18 in FIG. 7) in the bladder (or in the intermediate panel if one is used). The bladder may be pre-or post filled with the curable compound; that is, before or after the bladder is applied to the patient's body. The curable resin compound is preferably curable by irradiating the completed installation of the conformed bladder cast using an energy form selected from the group consisting of LED light curable, ultraviolet light, radio frequency energy, thermal energy, and ultra-sonic energy. Any of these energy forms may be emitted by a hand-held emitting device that is designed to emit the proper amount of energy to cure the resin in a reasonable time without risk of injury to the underlying tissues of the patient. A portable, stationary device may also be used. The type of energy source used will depend upon the exact nature of the curable compound used.

While one preferred embodiment of the invention involves the use of a curable, initially liquid resin compound, it will be understood that the curable compound can take a variety of forms including curable liquid, dry and curable clay compounds, as examples. See, for example, U.S. Pat. No. 4,683,877 (Ersfeld et al.) which describes a moisture-curable isolcyanate-functional, polyurethane pre-polymer including a tack-reducing agent. U.S. Pat. No. 6,030,355 (Callinan et al.) And U.S. Pat. No. 5,997,492 (Delmore et al.) describe other curable casting compounds, including silicate compounds, U.S. Pat. No, 4, 498,467 (Kirkpatrick et al.) discloses curable casting compounds, including polyoxyalkylene polyols, liquid isocyanates and urethane pre-polymers (used in a two-part room curable resin system). Although these previously described compounds are used in the cast forming arts, they are used in forming the cast substrate and not in a curable bladder, as would be used in the present invention.

The curable compound may also be one of the so-called “LED light curable” compounds. One example of a family member of this general class of compounds is the “LED 401 Adhesive” sold commercially by Master Bond™. This compound cures fully upon exposure to a 405 nm wavelength light source.

The elongated locking closure strip may be provided by first and second elongated strips of molded or extruded thermoplastic material, each first and second strip having the previously described hook-shaped cross section (at right angles to the longitudinal axis of the strip) to enable mutual engagement when attached to opposing edges of the bladder to be joined together as shown in FIG. 7, and previously described.

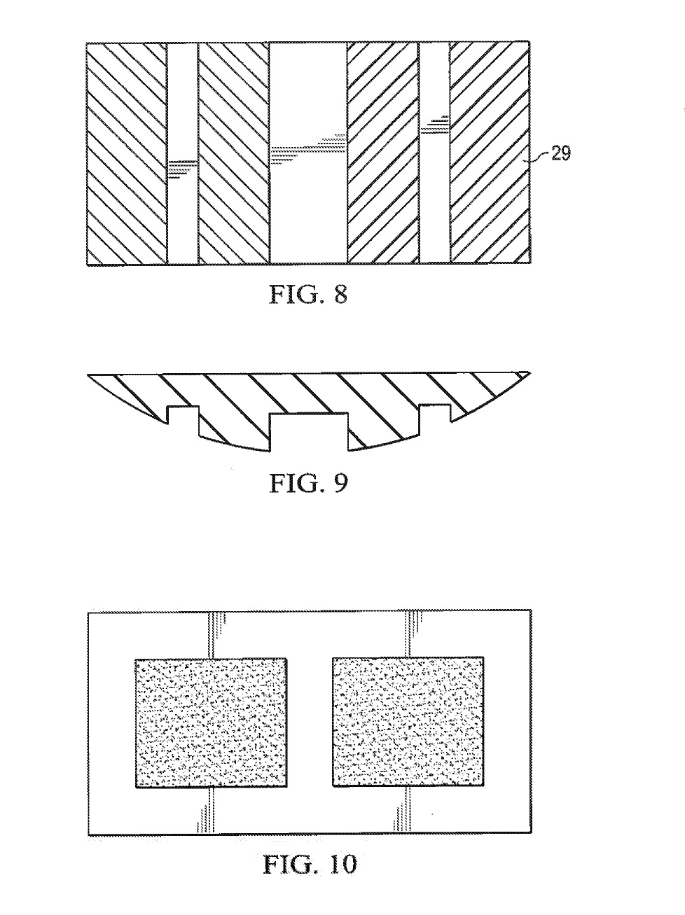

FIGS. 8, 9, and 10 illustrate respectively the tread surface (29 in FIG. 8), a cross section view (FIG. 9), and a top view (FIG. 10) of a walking sole that may be installed on the cast as needed. The sole may be made of natural or synthetic rubber. It may be attached to the plantar surface of the foot either before curing the curable compound inside the bladder or after curing is completed, depending on the particular circumstances.

To summarize, the process of forming a fully conformable, full contact orthopedic for a limb, appendage, or other body region requiring immobilization may thus include the following steps: applying any necessary wound dressing and a soft fabric sleeve such as a stockinette over the region to be enclosed in the cast; covering the sleeve with cast padding material, including felt strips as needed for structural support of the cast; providing a cast structure formed as a curable compound filled bladder; installing the bladder on the affected region of the patient's body; either pre- or post-installing a curable compound inside the bladder; and allowing the curable compound within the bladder to harden, preferably by applying a suitable energy source to effect the hardening. A walking sole may be included with casts formed on a patient's foot or lower leg.

While the invention has been shown in only one of its forms, it is not thus limited but is susceptible to various changes and modifications of the materials and process steps without departing from the spirit thereof. The embodiment described herein provides a cast according to the invention for a patient's lower leg, ankle, foot or other body part. Persons skilled in the art will recognize that the cast structure described may readily be adapted to other limbs applying the same basic concept of the curable resin-filled bladder that is individually shaped around the injured limb or appendage, or even as a body cast.

What is claimed is:

1. A fully conformable, total contact orthopedic cast for a limb or appendage requiring immobilization, comprising:
a bladder formed of an initially flexible, synthetic sheet material bonded around the perimeter of its edges to define a closed interior, the flexible sheet material allowing the bladder to be wrapped around the limb or appendage so that it closely conforms to the shape thereof;
a locking closure strip disposed along edges of the bladder to be joined from one end of the bladder to an opposite end thereof;
a curable compound, for filling the bladder to a predetermined level; wherein the bladder is wrapped around the appendage and the curable compound is subsequently cured.

2. The cast of claim 1, wherein the initially flexible bladder is pre- or post-filled with the curable compound.

3. The cast of claim 1, wherein the curable compound is selected from the group consisting of dry, liquid or clay type curable resins.

4. The cast of claim 1, wherein the bladder is equipped with a sealable opening for filling with a liquid curable resin.

5. A fully conformable orthopedic cast for a limb or appendage requiring immobilization, comprising:
a stockinette applied to the limb or appendage;
a fabric cast padding material applied over the stockinette;
a system of felt strips applied to the outside of the stockinette to provide structural form to the finished cast;
a bladder formed of an initially flexible sheet material bonded around the perimeter of its edges to form a closed interior, the initially flexible sheet material allowing the bladder to be wrapped around the limb or appendage so that it closely conforms to the shape thereof;
a locking closure strip disposed along edges of the bladder to be joined from one end of the bladder to its opposite end;
a curable compound filling the bladder to a predetermined level; wherein
the bladder is wrapped around the appendage over the felt strips and the stockinette and the curable compound is subsequently cured.

6. The cast of claim 5, wherein:
the initially flexible sheet material is a flexible, non-porous sheet capable of being hot-welded along the edges thereof using an ultrasonic or radio frequency heating process or using another type of seamless manufacturing process to create the closed interior.

7. The cast of claim 5, wherein the locking closure strip comprises:
first and second elongated strips of molded or extruded thermoplastic material, each first and second strip having a hook-shaped cross section (at right angles to the longitudinal axis of the strip) to enable mutual engagement when attached to opposing edges of the bladder to be joined together.

8. The cast of claim 7, wherein the locking closure strips are secured to the edges of the bladder using an ultrasonic or radio frequency heating process.

9. The cast of claim 5, wherein the liquid resin compound comprises:
a compound deposited within the bladder and formulated to harden when irradiated by an energy form selected from the group consisting of LED light, ultra-violet light, radio frequency energy, thermal energy, and ultra-sonic energy using a low energy hand-held device or portable, stationary device.

10. The cast of claim 2, wherein further comprising:
a secondary, elongated, intermediate bladder panel having first and second substantially parallel edges forming a tongue along and between first and second edges of the bladder and having first and second strips of the locking closure disposed along the first and second edges thereof.

11. The cast of claim 2, further comprising:
a sole formed of synthetic or natural rubber and attached to the plantar surface of the bladder of the cast formed around a patient's foot.

12. A method of forming a fully conformable, full contact orthopedic cast for a limb or appendage requiring immobilization, comprising the steps of:
providing a cast structure formed from an initially flexible bladder containing a curable compound, the flexible bladder filled with the curable compound being conformable to the shape of the affected limb or appendage of the body of a patient requiring immobilization;
installing the bladder cast;
curing the curable compound within the bladder to thereby form a hardened cast.

13. A method of forming a fully conformable, full contact orthopedic cast for a limb or appendage requiring immobilization, comprising the steps of:
applying any necessary wound dressing and a soft fabric sleeve such as a stockinette over the region to be enclosed in the cast;
covering the sleeve with cast padding material, including felt strips as needed for structural support of the cast;
providing a cast structure formed from an initially flexible bladder containing a curable compound, the flexible bladder filled with the curable compound being conformable to the shape of the affected limb or appendage of the body of a patient requiring immobilization;
installing the bladder cast;
curing the curable compound within the bladder to thereby form a hardened cast.

14. The method of claim 13, wherein the step of providing a cast structure comprises the steps of
forming the bladder of a synthetic sheet material bonded around the perimeter of its edges and having a sealable opening for filling with a liquid curable compound; and
providing a locking closure strip disposed along edges of the bladder to be joined from one end of the bladder to its opposite end.

15. The method of claim 13, wherein the step of installing the bladder cast comprises the steps of:
wrapping the bladder cast around the appendage to be enclosed in the cast;
providing a secondary, elongated, intermediate bladder panel having first and second substantially parallel edges forming a tongue along and between first and second edges of the bladder and having first and second strips of the locking closure disposed along the first and second edges thereof;
shaping the cast to conform the bladder cast to the enclosed appendage;
interlocking the locking closure; and
curing the curable compound within the bladder interior.

16. The method of claim 13, wherein the step of curing the curable compound includes the step of
irradiating the installed cast with an energy form selected from the group consisting of LED light, ultra-violet light, radio frequency energy, thermal energy, and ultra-sonic energy using a low energy hand-held device or portable stationary device.

17. The method of claim 16, where, if the cast is formed on a person's foot, comprising the step of
attaching a sole formed of synthetic or natural rubber to the plantar surface of the bladder formed around the person's foot.

18. The method of claim 13, wherein the locking closure strip can only be unlocked through the use of an appropriate cast removal key or tool in order to better insure that the patient may not remove or adjust or otherwise alter the full contact fit of the cast.
